(19) United States
(12) Patent Application Publication
(10) Pub. No.: US 2024/0024293 A1
(43) Pub. Date: Jan. 25, 2024

(54) NOMETHIAZOLES AS A TREATMENT FOR RETT SYNDROME

(71) Applicant: REVIVO THERAPEUTICS, DURHAM, NC (US)

(72) Inventors: Doug COWART, Williamsburg, VA (US); Craig HARTMAN, Loveland, CO (US)

(21) Appl. No.: 17/427,351

(22) PCT Filed: Feb. 3, 2020
(86) PCT No.: PCT/US20/16406

§ 371 Date: Feb. 2, 2022

Related U.S. Application Data
(60) Provisional application No. 62/800,021, filed on Feb. 1, 2019.

(51) Int. Cl. A61K 31/426; A61P 25/00

(52) U.S. Cl. CPC A61K 31/426; A61P 25/00

(57) ABSTRACT

The present application relates to methods for treatment of Rett syndrome comprising administering an effect amount of nomethiazoles to a subject need thereof.

### RELATED APPLICATIONS

This application claims priority to U.S. Provisional Application No. 62/800,021, filed Feb. 1, 2019. The entire contents of this patent application are hereby incorporated herein by reference.

### FIELD OF THE INVENTION

The disclosure provides methods of treating Rett syndrome using nomethiazoles.

### BACKGROUND OF THE INVENTION

Rett syndrome is an X-linked neurological disorder that is typically first recognized in infancy and seen almost always in girls. It is often misdiagnosed as autism, cerebral palsy, or non-specific developmental delay, and has a high prevalence of 1 in 10,000 female births. It is characterized by progressive development of motor and neurological dysfunction. Patients affected with Rett syndrome exhibit symptoms including communication skill regression and loss of movement skills after birth. Other symptoms include: seizures; disorganized breathing patterns while awake; scoliosis; and sleep disturbances. These problems can affect learning, speech, mood, movement, cardiac function, chewing, swallowing and digestion.

Most patients with Rett syndrome carry a mutation in the MECP2 gene. Study in mutant mice shows that expression of Mecp2 alleviates symptoms of Rett syndrome and these mice further regain normal movements. These findings suggest that MECP2 is required for maintenance of neurons after birth. Therefore, Rett syndrome may be reversed by pharmacological means after symptoms onset.

Currently, there is no approved therapies for Rett syndrome. Thus, there is a critical need for the development of new therapies for Rett syndrome.

### SUMMARY

The present disclosure provides, inter alia, a method of treating Rett syndrome in a subject in need thereof, the method comprising administering to the subject a therapeutically effective amount of a nomethiazole having the structure:

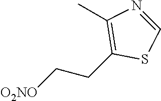

or a pharmaceutically acceptable salt thereof.

In another embodiment, provided herein is a method of treating Rett syndrome in a subject in need thereof, the method comprising administering to the subject a therapeutically effective amount of 2-(4-methylthiazol-5-yl)ethyl nitrate maleate salt.

### BRIEF DESCRIPTION OF THE DRAWINGS

FIG. 1 shows an XRPD graphic scan of 2-(4-methylthiazol-5-yl)ethyl nitrate maleate salt.

FIG. 2 shows startle response tests in mutant and wildtype 8-week old mice treated with 2-(4-methylthiazol-5-yl)ethyl nitrate maleate salt.

### DETAILED DESCRIPTION

The present invention provides a method of treating Rett syndrome in a subject in need thereof, the method comprising administering to the subject a therapeutically effective amount of a compound having the structure:

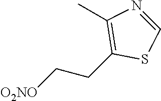

or a pharmaceutically acceptable salt thereof.

In some embodiments, the compound is the maleate salt.

In some embodiments, the maleate salt is in crystalline form.

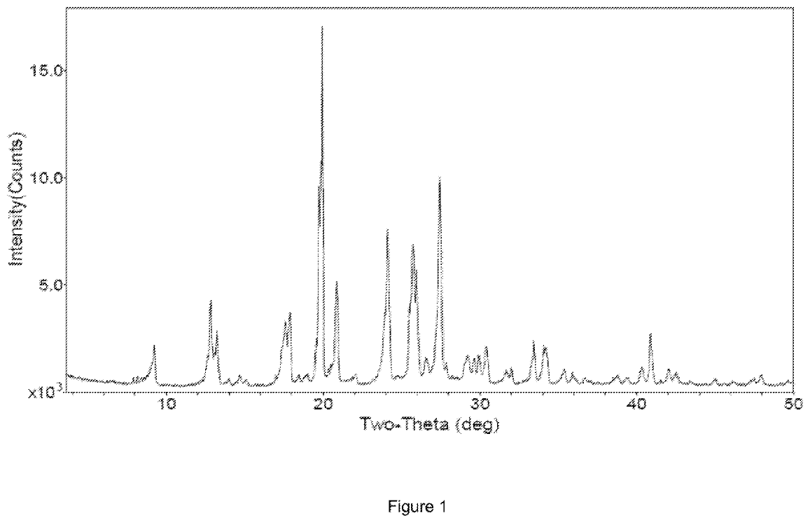

In some embodiments, the crystal form has the XRPD graphic scan of FIG. 1.

In some embodiments, the compound is formulated in a sustained release formulation.

In yet another embodiment of the invention, the said subject is administered from about 0.5 mg/day to about 3000 mg/day of the compound or a pharmaceutically acceptable salt thereof.

In some embodiments, the dosage is administered at least every other day or once a day.

In some embodiments, wherein the method comprises administering a pharmaceutical composition comprising about 0.5 mg to about 3000 mg of the compound or a pharmaceutically acceptable salt thereof, and a pharmaceutically acceptable carrier.

In some embodiments, wherein the patient is administered from about 0.5 mg to about 3000 mg of the compound or a pharmaceutically acceptable salt thereof in a 24-hour period.

In some embodiments, wherein the compound or a pharmaceutically acceptable salt thereof is administered orally, intravenously, intraperitoneally, parenterally, rectally, enterally, transdermally or transmucosally.

In one aspect, the invention provides a method of treating a subject that has a mutation in a gene encoding methyl CpG-binding protein 2 (MECP2).

In some embodiments, the subject acquires neuroprotection.

In some embodiments, the subject's neuronal plasticity and memory is enhanced.

In some embodiments, the subject's loss of MeCP2 reduced NO availability is corrected.

In some embodiments, NO/cGMP signaling is activated.

In some embodiments, peripheral NO signaling is enhanced.

In some embodiments, GABA signaling is upregulated.

In some embodiments, glutamate excitotoxicity in CNS is reduced.

In some embodiments, anti-inflammation is initiated.

In some embodiments, CREB signaling is enhanced.

In some embodiments, the cGMP/CREB pathway is activated.

In some embodiments, CREB signaling is targeted.

In some embodiments, the targeted CREB signaling causes synaptic repair.

In some embodiments, the targeted CREB signaling causes neurogenesis.

In some embodiments, Mecp2 expression in GABAergic neurons is restored.

In some embodiments, phosphorylation of CREB is increased.

In some embodiments, the forebrain function is enhanced.

It is further appreciated that certain features of the invention, which are, for clarity, described in the context of separate embodiments, can also be provided in combination in a single embodiment (while the embodiments are intended to be combined as if written in multiply dependent form). Conversely, various features of the invention which are, for brevity, described in the context of a single embodiment, can also be provided separately or in any suitable subcombination. Thus, it is contemplated as features described as embodiments of the compounds of the present disclosure can be combined in any suitable combination.

The term “compound” as used herein is meant to include all stereoisomers, geometric isomers, tautomers and isotopes of the structure depicted. The term is also meant to refer to a compound of the invention, regardless of how they are prepared, e.g., synthetically, through biological process (e.g., metabolism or enzyme conversion), or a combination thereof.

All compound, and pharmaceutically acceptable salts thereof, can be found together with other substances such as water and solvents (e.g., hydrates and solvates) or can be isolated. When in the solid state, the compound described herein and salts thereof may occur in various forms and may, e.g., take the form of solvates, including hydrates. The compound may be in any solid-state form, such as a polymorph or solvate, so unless clearly indicated otherwise, reference in the specification to compounds and salts thereof should be understood as encompassing any solid-state form of the compound.

In some embodiments, the compound of the invention, or salts thereof, is substantially isolated. By “substantially isolated” is meant that the compound is at least partially or substantially separated from the environment in which it was formed or detected. Partial separation can include, e.g., a composition enriched in the compounds of the invention. Substantial separation can include compositions containing at least about 50%, at least about 60%, at least about 70%, at least about 80%, at least about 90%, at least about 95%, at least about 97%, or at least about 99% by weight of the compounds of the invention, or salt thereof.

The phrase “pharmaceutically acceptable” is employed herein to refer to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present invention also includes pharmaceutically acceptable salts of the compounds described herein. The term “pharmaceutically acceptable salts” refers to derivatives of the disclosed compound wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the non-toxic salts of the parent compound formed, e.g., from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol or butanol) or acetonitrile (MeCN) are preferred. Lists of suitable salts are found in Remington's Pharmaceutical Sciences, 17^{th} Ed., (Mack Publishing Company, Easton, 1985), p. 1418, Berge et al., J. Pharm. Sci., 1977, 66(1), 1-19 and in Stahl et al., Handbook of Pharmaceutical Salts: Properties, Selection, and Use, (Wiley, 2002). In some embodiments, the compounds described herein include the N-oxide forms.

### Rett Syndrome and Nomethiazoles

Mutations in the coding sequence of the X-linked gene MeCP2 (Methyl CpG—binding protein) are present in around 80% of patients with Rett Syndrome, a common cause of intellectual disability in females and to date without any effective pharmacological treatment. Surprisingly by modulating downstream effector molecules which are deregulated in this disease, nomethiazoles provide for an effective amelioration of the progression of this disease in animal models and humans.

Chemical compounds within the specific class of nomethiazoles reduce the progression of RETT Syndrome. Nomethiazoles are described in the paper “Design and synthesis of neuroprotective methylthiazoles and modification as NO-chimeras for neurodegenerative therapy” (J Med Chem. 2012 Aug. 9; 55(15):6784-801), which is incorporated by reference in its entirety.

To investigate the relationship between loss of MeCP2 and clinical aspects of this disease, MeCP2 null mouse model(s) has been utilized for mechanistic studies of the utility of nomethiazoles to ameliorate certain aspects of this disease state. Nomethiazoles administered to these specific mouse models utilized for evaluation of therapies for RETT Syndrome have provided evidence for therapeutic benefit in the treatment of this genetic disorder. This therapeutic benefit is manifested by epigenetic effects yielding a decrease in the progression of the clinical findings associated with this disease. Findings suggest that there are several mechanisms responsible for the beneficial effects of nomethiazoles in this disease state. The effects of this class of therapeutic agent is novel and due to any of the following mechanisms or the convergence of more than one of the following outlined mechanisms. The following list of mechanisms or other similar mechanisms are anticipated to underlie the observed beneficial effects.

A relevant, and so far, unexplored, feature of RTT patients, is a marked reduction in peripheral and the neuronal circulation. Such functional aspects are associated with an intravascular increase in superoxide anion production. Nomethiazoles improve the reduced endothelium-dependent relaxation observed in this disease state, by increasing nitric oxide (NO) availability in the central nervous system and in the peripheral compartment. These vascular alterations are reversed by administration of specific nomethiazoles during sub chronic oral or parenteral treatment restoring endothelial NO signaling and decreasing intravascular ROS production normalizing vascular eNOS gene expression. The vascular effects of nomethiazoles attenuate the prominent neurological symptoms in animal models and in children by reversing hypo-perfusion in the area of midbrain and upper brainstem, and concomitantly improving poor peripheral circulation. Nomethiazoles also reduce the cyanosis of the extremities in RETT syndrome models and in human disease thus decreasing the morbidity of this disease.

Elevated glutamate levels are observed in RETT models. Nomethiazoles alter microglial glutamate release and abolish or reduce the neurotoxic activity of glutamate, known to be elevated in Mecp2-null microglia by inhibiting glutamate production and/or release. Microglial abnormalities appear to influence the onset and progression of RTT and attenuation of microglia glutamate synthesis or release may fully or partially mediate the beneficial effects of nomethiazoles seen in RETT syndrome animal models and humans.

Methiazole molecular structural components of the nomethiazole class act to reduce excitotoxicity of neurons. This effect of methiazoles modulates excessive release of intracellular calcium, which is known to increase cellular metabolic stress. This effect is due to specific effects on neurotransmitter release, which is modulated by effects of methiazoles on excitatory neurons in the CNS. Some or all the effect is modulated by a specific allosteric GABAminergic activity of the nomethiazole class, which dampens the neurotoxic excessive neuronal activity that is a hallmark in this disease.

Specific inflammatory cytokines are down regulated by nomethiazoles, thereby reducing the neuronal inflammation that leads to a cascade of neurotoxicity in Rett Syndrome. Specific actions of nomethiazoles to reduce specifically identified cytokines pay a role in the utility of this class in RETT syndrome

CREB and Phosphorylated CREB (messengers of cAMP response element binding protein signaling) are upregulated by nomethiazoles and can alleviate RTT phenotypes both in vitro and in vivo. Nomethiazole activation/phosphorylation of CREB directly regulates the expression of specific genes (e.g., c-Fos, Arc, BDNF, and Wnt2), which are involved in RETT Syndrome.

Administration of specific plasma levels of nomethiazoles with exposure for specific periods of time using specific mechanisms of delivery are critical to the beneficial effects of nomethiazoles seen in RETT Syndrome. Specific formulations for oral or parenteral administration are required for clinical utility.

### Nomethiazole for Treating Rett

The nomethiazole 2-(4-methylthiazol-5-yl)ethyl nitrate has the following structure:

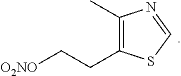

Provided herein is a method of treating Rett syndrome in a subject in need thereof, the method comprising administering to the subject a therapeutically effective amount of the nomethiazole 2-(4-methylthiazol-5-yl)ethyl nitrate, or a pharmaceutically acceptable salt thereof.

The compound 2-(4-methylthiazol-5-yl)ethyl nitrate is known to interact with amino acid neurotransmitter receptors such as the NMDA receptor and the γ-aminobutyric acid type A (GABA A) receptor. This compound is also known to stimulate cerebral soluble guanylyl cyclase (GCase). As such, this compound is useful for its neuroprotective properties, and effecting cognition enhancement. See, e.g., U.S. Pat. Nos. 6,310,052 and 9,114,135, both of which are incorporated herein by reference in their entireties. It has been found that new solid forms of 2-(4-methylthiazol-5-yl)ethyl nitrate can be prepared as the maleate salt form. As shown in U.S. Pat. No. 9,114,135, this salt form exhibits new physical properties that can be exploited in order to achieve new properties, making it useful as a drug substance.

This compound can be synthesized by methods as described in U.S. Pat. Nos. 5,883,122; 6,310,052, and 9,114,135. Various compounds for use in the methods of the invention are commercially available and/or can be synthesized by standard techniques. In general, nitrate esters can be prepared from the corresponding alcohol, oxirane or alkene by standard methods, that include: nitration of alcohols and oxiranes, mixed aqueous/organic solvents using mixtures of nitric and sulfuric acid and/or their salts, with temperature control; nitration of alcohols and oxiranes in acetic anhydride using nitric acid or its salts with or without added acid catalyst, with temperature control; nitration of an alcohol with a nitronium salt, e.g. a tetrafluoroborate; nitration of an alkene with thallium nitrate in an appropriate solvent.

### Methods of Treatment

The compound of the present disclosure can have novel mechanism of action by acting as dual acting pharmacophore. For example, the compound can act as GABA B allosteric modulator (etomidate receptor) and as a weak GABA A agonist, in part, because GABA A partial agonists have anxiolytic activity.

In some embodiments, the compound of the disclosure is an NO-cGMP activating procognitive/anxiolytic dual pharmacophore. For example, the compound activates NO/cGMP signaling.

In some embodiments, the compound of the present disclosure can alter Rett syndrome by several pharmacological mechanisms. In another embodiment, the compound can alter CREB signaling. In another embodiment, the compound can alter GABA signaling. In another embodiment, the compound can enhanced peripheral NO signaling. In yet another embodiment, the compound can lead to reduction of glutamate excitotoxicity in CNS via administering treatments upregulating GABA. In yet another embodiment, the compound can lead to anti-inflammatory effects.

In some embodiments, the compound of the present disclosure can treat Rett syndrome by affecting CREB. In another embodiment, the compound can enhance CREB signaling to treat Rett syndrome. In another embodiment, the compound can activate the cGMP/CREB pathway. In another embodiment, the compound can target CREB signaling causing synaptic repair. In another embodiment, the compound can target CREB signaling causing neurogenesis. In another embodiment, the compound and potential for disease modification.

In some embodiments, the compound of the present disclosure can treat Rett syndrome by altering GABA signaling. In another embodiment, the compound can lead to restoration of Mecp2 expression in GABAergic neurons.

In some embodiments, the compound of the present disclosure can treat Rett syndrome by oral, i.p., or i.v. administration. In another embodiment, the compound can be administered by oral administration. In another embodiment, the compound can be administered by i.p. administration. In another embodiment, the compound can be administered by i.v. administration.

In some embodiments, the compound is a GABA allosteric modulator. In some embodiments, the compound increases phosphorylation of CREB and enhances forebrain function. In another embodiment, the compound is neuroprotective in neuro-development models and may provide protection in Rett models. In another embodiment, the compound enhances neuronal plasticity and memory in Rett models. In another embodiment, the compound can correct the loss of MeCP2 reduced NO availability.

As used herein, the term “individual,” “subject,” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent such as an amount of any of the solid forms or salts thereof as disclosed herein that elicits the biological or medicinal response in a tissue, system, animal, individual or human that is being sought by a researcher, veterinarian, medical doctor or other clinician. Particular reference is made to an amount that can ameliorate biochemical and functional abnormalities associated with loss-of-function mutations of the gene encoding methyl-CpG binding protein 2 (MeCP2). It is to be understood that a therapeutically effective amount of an agent or combinatory therapy may vary according to factors such as the disease state, age, and weight of the subject, and the ability of the agent to elicit a desired response in the subject. Dosage regimens may be adjusted to provide the optimum therapeutic response. A therapeutically effective amount is also one in which any toxic or detrimental effects of the active compound are outweighed by the therapeutically beneficial effects. An appropriate “effective” amount in any individual case may be determined using techniques known to a person skilled in the art.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit/risk ratio.

As used herein, the phrase “pharmaceutically acceptable carrier or excipient” refers to a pharmaceutically-acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use. In one embodiment, each component is “pharmaceutically acceptable” as defined herein. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

As used herein, the term “treating” or “treatment” refers to inhibiting a disease; for example, inhibiting a disease, condition, or disorder in an individual who is experiencing or displaying the pathology or symptomology of the disease, condition, or disorder (i.e., arresting further development of the pathology and/or symptomology) or ameliorating the disease; for example, ameliorating a disease, condition, or disorder in an individual who is experiencing or displaying the pathology or symptomology of the disease, condition, or disorder (i.e., reversing the pathology and/or symptomology) such as decreasing the severity of the disease.

The term “prevent,” “preventing,” or “prevention” as used herein, comprises the prevention of at least one symptom associated with or caused by the state, disease or disorder being prevented.

It is appreciated that certain features of the invention, which are, for clarity, described in the context of separate embodiments, can also be provided in combination in a single embodiment (while the embodiments are intended to be combined as if written in multiply dependent form). Conversely, various features of the invention which are, for brevity, described in the context of a single embodiment, can also be provided separately or in any suitable subcombination.

### Formulation, Dosage Forms and Administration

When employed as pharmaceuticals, the compound of the present disclosure can be administered in the form of pharmaceutical compositions. Thus, the present disclosure provides a composition comprising a compound as recited in any of the claims and described herein, or a pharmaceutically acceptable salt thereof, or any of the embodiments thereof, and at least one pharmaceutically acceptable carrier or excipient. These compositions can be prepared in a manner well known in the pharmaceutical art, and can be administered by a variety of routes, depending upon whether local or systemic treatment is indicated and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, e.g., by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This invention also includes pharmaceutical compositions which contain, as the active ingredient, the compound of the present disclosure or a pharmaceutically acceptable salt thereof, in combination with one or more pharmaceutically acceptable carriers or excipients. In some embodiments, the composition is suitable for topical administration. In making the compositions of the invention, the active ingredient is typically mixed with an excipient, diluted by an excipient or enclosed within such a carrier in the form of, e.g., a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, e.g., up to 10% by weight of the active compound, soft and hard gelatin capsules, suppositories, sterile injectable solutions and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

The compounds of the invention may be milled using known milling procedures such as wet milling to obtain a particle size appropriate for tablet formation and for other formulation types. Finely divided (nanoparticulate) preparations of the compounds of the invention can be prepared by processes known in the art see, e.g., WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup and methyl cellulose. The formulations can additionally include: lubricating agents such as talc, magnesium stearate and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

In some embodiments, the pharmaceutical composition comprises silicified microcrystalline cellulose (SMCC) and at least one compound described herein, or a pharmaceutically acceptable salt thereof. In some embodiments, the silicified microcrystalline cellulose comprises about 98% microcrystalline cellulose and about 2% silicon dioxide w/w.

In some embodiments, the composition is a sustained release composition comprising at least one compound described herein, or a pharmaceutically acceptable salt thereof, and at least one pharmaceutically acceptable carrier or excipient. In some embodiments, the composition comprises at least one compound described herein, or a pharmaceutically acceptable salt thereof, and at least one component selected from microcrystalline cellulose, lactose monohydrate, hydroxypropyl methylcellulose and polyethylene oxide. In some embodiments, the composition comprises at least one compound described herein, or a pharmaceutically acceptable salt thereof, and microcrystalline cellulose, lactose monohydrate and hydroxypropyl methylcellulose. In some embodiments, the composition comprises at least one compound described herein, or a pharmaceutically acceptable salt thereof, and microcrystalline cellulose, lactose monohydrate and polyethylene oxide. In some embodiments, the composition further comprises magnesium stearate or silicon dioxide. In some embodiments, the microcrystalline cellulose is Avicel PH102™. In some embodiments, the lactose monohydrate is Fast-flo 316™. In some embodiments, the hydroxypropyl methylcellulose is hydroxypropyl methylcellulose 2208 K4M (e.g., Methocel K4 M Premier™) and/or hydroxypropyl methylcellulose 2208 K100LV (e.g., Methocel K00LV™). In some embodiments, the polyethylene oxide is polyethylene oxide WSR 1105 (e.g., Polyox WSR 1105™)

In some embodiments, the compounds of the present invention can be prepared in a microbead formulation. In some embodiments, the microbead formulation can be administered orally. In some embodiments, the microbead formulation can be administered as capsules or tablets. In some embodiments, the microbead formulation can be administered by adding to food. In some embodiments, the capsules can be administered by adding to food.

In some embodiments, a wet granulation process is used to produce the composition. In some embodiments, a dry granulation process is used to produce the composition.

The compositions can be formulated in a unit dosage form, each dosage containing from about 5 to about 1,000 mg (1 g), more usually about 100 mg to about 500 mg, of the active ingredient. In some embodiments, each dosage contains about 10 mg of the active ingredient. In some embodiments, each dosage contains about 50 mg of the active ingredient. In some embodiments, each dosage contains about 25 mg of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The components used to formulate the pharmaceutical compositions are of high purity and are substantially free of potentially harmful contaminants (e.g., at least National Food grade, generally at least analytical grade, and more typically at least pharmaceutical grade). Particularly for human consumption, the composition is preferably manufactured or formulated under Good Manufacturing Practice standards as defined in the applicable regulations of the U.S. Food and Drug Administration. For example, suitable formulations may be sterile and/or substantially isotonic and/or in full compliance with all Good Manufacturing Practice regulations of the U.S. Food and Drug Administration.

The active compound may be effective over a wide dosage range and is generally administered in a therapeutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms and the like.

The therapeutic dosage of a compound of the present invention can vary according to, e.g., the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral administration. Some typical dose ranges are from about 1 vg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, e.g., about 0.1 to about 1000 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, e.g., liquid paraffin, polyoxyethylene alkyl ether, propylene glycol, white Vaseline, and the like. Carrier compositions of creams can be based on water in combination with glycerol and one or more other components, e.g., glycerinemonostearate, PEG-glycerinemonostearate and cetylstearyl alcohol. Gels can be formulated using isopropyl alcohol and water, suitably in combination with other components such as, e.g., glycerol, hydroxyethyl cellulose, and the like. In some embodiments, topical formulations contain at least about 0.1, at least about 0.25, at least about 0.5, at least about 1, at least about 2 or at least about 5 wt % of the compound of the invention. The topical formulations can be suitably packaged in tubes of, e.g., 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers or stabilizers will result in the formation of pharmaceutical salts.

The therapeutic dosage of a compound of the present invention can vary according to, e.g., the particular use for which the treatment is made, the manner of administration of the compound, the health and condition of the patient, and the judgment of the prescribing physician. The proportion or concentration of a compound of the invention in a pharmaceutical composition can vary depending upon several factors including dosage, chemical characteristics (e.g., hydrophobicity), and the route of administration. For example, the compounds of the invention can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The following Examples further illustrate the present invention and are not intended to be limiting in any respect. Those skilled in the art will recognize or be able to ascertain using no more than routine experimentation, numerous equivalents to the specific procedures described herein. Such equivalents are considered to be within the scope of this invention and are covered by the claims.

### EXAMPLES

The examples in this section are offered by way of illustration and not by way of limitation.

The following abbreviations may be used herein: MS (Mass spectrometry); Me (methyl); mg (milligram(s)); min. (minutes(s)); mL (milliliter(s)); mmol (millimole(s)); N (normal); nM (nanomolar); NMR (nuclear magnetic resonance spectroscopy); r.t. (room temperature), μg (microgram(s)); μL (microliter(s)); μM (micromolar); wt % (weight percent). RTT (Rett syndrome); NO (nitric oxide); CREB (cAMP response element-binding protein); GABA (gamma-aminobutyric acid); cAMP (Cyclic adenosine monophosphate); ms (millisecond(s)).

### Example 1: Preparation RIV-5061

The synthesis of RIV-5061, also known as 2-(4-methylthiazol-5-yl)ethyl nitrate maleate (NMZM), can be found in U.S. Pat. No. 9,114,135, which is incorporated herein by reference in its entirety. The synthetic route employed for synthesis of RIV-5061 is shown below:

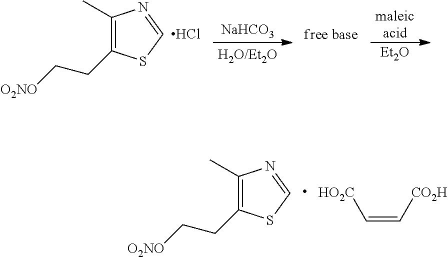

Spectral and physical properties were in agreement with those previously reported in U.S. Pat. No. 9,114,135. A typical X-Ray Powder Diffraction (XRPD) graphic scan of 2-(4-methylthiazol-5-yl)ethyl nitrate maleate salt is shown in FIG. 1.

### Example 2: Preparation of RETT Mouse Model

Experiments employed wild type (WT) mice and Mecp2 female mice (HET) that were provided by The Jackson Laboratory, Bar Harbor, ME. These Mecp2 mice exhibit Rett syndrome-like neurological defects.

Breeding of the experimental mice were done at The Jackson Laboratories and were shipped at approximately 5 weeks of age. Dosing experiments were conducted at approximately 6 weeks of age.

Treatment Groups (N=20) were as follow:
WT—Vehicle
HET—Vehicle
HET—RIV-0561 (1.35 mg/kg; ip QD)+0.675 mg/ml of RIV-0561 in drinking water
HET—RIV-0561 (13.5 mg/kg; ip QD)+0.675 mg/ml of RIV-0561 in drinking water

NeuroCube® and Startle experiments were conducted when mice were at 8 and 12 weeks of age. All tests were conducted within 2 hours post IP dosing.

### Example 3: Startle Response

The acoustic startle measures an unconditioned reflex response to external auditory stimulation. Mice were placed in the startle enclosures and secured in the sound-attenuated startle chamber (Med Associates Inc., St Albans, VT) on top of a force transducer plate that measures the force of the movements made by the mouse for a 5 min habituation period of white noise (70 dB). Subsequent test sessions consisted of 10 blocks of eleven trials each. Within each block, stimuli of 70, 75, 80, 85, 90, 95, 100, 105, 110, 115, and 120 dB were presented in a random order with a variable inter-trial interval of mean 15 sec (10-20 sec). The duration of the stimulus was 40 ms. Responses were recorded for 150 ms from startle onset and were sampled every ms. Mice were returned to their home cage immediately after testing, and the startle chambers and enclosures were cleaned between test sessions.

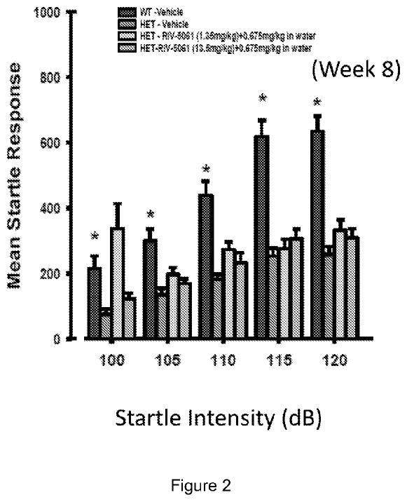

### Results: Startle Response at 8 Weeks (FIG. 2)

HET mice show significantly lower startle response at 8 weeks of age. RIV-5061 showed a tendency to improve startle response in HET mice at 8 weeks at all doses tested. This general finding suggests improvement in neurological status and anxiety in HET animals treated with RIV-5061.

### Example 4: Gait Analysis Method—NeuroCube®

NeuroCube® system is a proprietary gait analysis system. The platform employs computer vision to detect changes in gait geometry and gait dynamics in rodent models of neurological disorders, pain, and neuropathies. This platform is unique for gait testing for the following reasons: (1) it is completely automated and thus removes any bias or subjectivity; (2) the system captures both gait geometry and gait dynamics (stance, swing, propulsion, etc.); and (3) the sensitivity of the computer vision and bioinformatics allow the capture of symptoms of the disease model earlier and more accurately.

Mice are placed in the NeuroCube® for a 5-min test. The most dominant features that define the disease phenotype (symptom descriptors) are identified and ranked. Complex bioinformatics algorithms are employed to calculate the discrimination probability between WT and HET mice and can thus detect a test compound's ability to reverse the disease phenotype.

### Results: Gait Features at 12 Weeks (Table 1):

RIV-5061 improved important gait features in RETT mice (up to >60%) after 12 weeks of treatment, particularly at the lower dose (1.35 mg/kg IP daily+0.675 mg/ml of RIV-0561 in drinking water). These results are shown in Table 1.

TABLE 1

| | % Discrimination | % Recovery |
|---|---|---|
| Gait Features at 12 weeks | WT vs HET Vehicle | RIV-5061 |
| Overall Gait | 93%* | 24% |
| Gait Dynamic and Geometry | 75%* | 53% |
| Paw Features | 66%* | 63% |
| Rhythmicity | 70%* | 0% |
| Body Motion | 95%* | 19% |
| Paw Positioning | 65%* | 17% |

*p < 0.01 compared to WT vehicle

The experimental results show the importance of Gait in Rett syndrome. At a clinical level, gait in children with RETT syndrome is characterized by ataxia, apraxia and spasticity with and without clonus. Affected girls develop a preference for one leg, putting it forward at every step as the foremost leg, using the contralateral one just for support and balance. Based on available data, 20-40% children with RTT will never be able to walk. Furthermore, of the girls who gain the ability to walk, up to 80% might lose it along with disease progression Gait disturbance is one of one of the most life burdening symptoms, in RTT.

### Discussion

The spectrum of pharmacological activity of RIV-5061 from preclinical studies has suggested that it may pose some benefit in the treatment of Rett syndrome. Prior Human Experience with this class of nomethiazole molecules (RIV-1061) suggest that RIV-5061 is a potentially clinically well tolerated therapy and may be more therapeutically advantageous if administered in a sustained release formulation. The use of RIV-5061 may provide through its procognitive, anti-inflammatory and anxiolytic effects a useful treatment for this debilitating neurodevelopment syndrome.

Existing data suggests that there is a beneficial therapeutic window for RIV-5061 plasma concentrations that may be addressed by specific formulations of RIV-5061. Specific Domains of functional testing have identified activity of RIV-5061 in the RETT mouse Model that may indicate its potential therapeutic utility in the treatment of RETT Syndrome. The findings are anticipated to be generalizable to both females and males with RETT symptomology.

What is claimed is:

1. A method of treating Rett syndrome in a subject in need thereof, the method comprising administering to the subject a therapeutically effective amount of a compound having the structure: 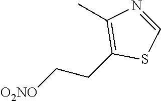
or a pharmaceutically acceptable salt thereof.

2. The method of claim 1, wherein the compound is the maleate salt.

3. The method of claim 2, wherein the maleate salt is in crystalline form.

4. The method of claim 3, wherein the crystal form has the XRPD graphic scan of FIG. 1.

5. The method of any one of claims 1-4, wherein the compound is formulated in a sustained release formulation.

6. The method of any one of claims 1-5, wherein the said subject is administered from about 0.5 mg/day to about 3000 mg/day of the compound or a pharmaceutically acceptable salt thereof.

7. The method of any one of claims 1-6, wherein said dosage is administered at least every other day or once a day.

8. The method of any one of claims 1-5, wherein the method comprises administering a pharmaceutical composition comprising about 0.5 mg to about 3000 mg of the compound or a pharmaceutically acceptable salt thereof, and a pharmaceutically acceptable carrier.

9. The method of any one of claims 1-6, wherein the patient is administered from about 0.5 mg to about 3000 mg of the compound or a pharmaceutically acceptable salt thereof in a 24-hour period.

10. The method of any one of claims 1-9, wherein the compound or a pharmaceutically acceptable salt thereof is administered orally, intravenously, intraperitoneally, parenterally, rectally, enterally, transdermally or transmucosally.

11. The method of claim 1, wherein the subject has a mutation in a gene encoding methyl CpG-binding protein 2 (MECP2).

12. The method of claim 1, wherein the subject acquires neuroprotection.

13. The method of claim 1, wherein the subject's neuronal plasticity and memory is enhanced.

14. The method of claim 1, wherein the subject's loss of MeCP2 reduced NO availability is corrected.

15. The method of claim 1, wherein NO/cGMP signaling is activated.

16. The method of claim 1, wherein peripheral NO signaling is enhanced.

17. The method of claim 1, wherein GABA signaling is upregulated.

18. The method of claim 17, wherein glutamate excitotoxicity in CNS is reduced.

19. The method of claim 1, wherein anti-inflammation is initiated.

20. The method of claim 1, wherein CREB signaling is enhanced.

21. The method of claim 1, wherein the cGMP/CREB pathway is activated.

22. The method of claim 1, wherein CREB signaling is targeted.

23. The method of claim 22, wherein the targeted CREB signaling causes synaptic repair.

24. The method of claim 22, wherein the targeted CREB signaling causes neurogenesis.

25. The method of claim 1, wherein Mecp2 expression in GABAergic neurons is restored.

26. The method of claim 1, wherein phosphorylation of CREB is increased.

27. The method of claim 26, wherein the forebrain function is enhanced.
